(19) United States
(12) Patent Application Publication
(10) Pub. No.: US 2004/0153151 A1
(43) Pub. Date: Aug. 5, 2004

(54) Breast prosthesis

(76) Inventor: Ricardo Gonzales de Vicente, Valencia (ES)

Correspondence Address: SANCHELIMA & ASSOCIATES, P.A., JESUS SANCHELIMA, ESQ., 235 S.W. LE JEUNE ROAD, MIAMI, FL, 33134

(21) Appl. No.: 10/476,918

(22) PCT Filed: Apr. 25, 2002
(86) PCT No.: PCT/ES02/00202

(30) Foreign Application Priority Data
May 7, 2001 (ES) P 0101038

(51) Int. Cl. A61F 2/12

(52) U.S. Cl. 623/8

(57) ABSTRACT

A breast prosthesis which, using a widely known material such as silicon, establishes a trabecular body (1) or one of micro-cells (3), with or without a covering (2), provides a prosthesis eliminating the risks of puncture as well as extravasation of the inner fluid, being possible to obtain a lower density than conventional ones, and therefore a lower weight, also eliminating problems due to sagging.

### OBJECT OF THE INVENTION

The new breast prosthesis proposed uses a widely used and accepted material for these implants, such as silicon, but with a body with a trabecular or micro-cell structure, either wrapped in a silicon covering or not, providing a lighter body with variable density with no liquid or gelatin components that can extravasate the limits of the coverings, leaking out, furthermore eliminating the risks of punctures, encapsulations, etc.

### BACKGROUND OF THE INVENTION

To date, implanted breast prostheses essentially consist of two components: an outer one or a covering and an inner one or the filling. The outer one is internationally common for all trademarks and manufacturers of breast implants and is based on a vulcanized silicon covering of two, three or, in the best of cases, five layers, for the purpose of trying to decrease to a minimum the “migration” or transudation phenomenon of the implant, which consists of the implant filling product or substance (silicon, saline, hydrogel, etc.) passing through microscopic pores of the wall of the covering, with more or less success in each case.

The main drawback with traditional implants is that this migration results in a decrease of the initial volume of the patient's implant and, therefore, of the volume obtained after her operation in some cases or the total loss of volume in the case of implants with a filling valve mechanism. In the case of silicon gel implants, the silicon can migrate through different routes: fat, lymph nodes, etc., with unwanted results for the organism.

Some breast prostheses, when they are crushed by body weight such as when sleeping, with time end up losing volume, and therefore losing the reason for which they were implanted.

All current implants have a liquid or gel content that can extravasate upon bursting, they are also susceptible to punctures when handled for the implanting or subsequent use thereof due to fatigue of the material with time, due to friction between folds and creases, etc., with the subsequent economic losses for the distributor, doctor, and the drawbacks for the patient in terms of economic drawbacks as well as discomforts upon second operations, touch-ups, etc. This effect being acute when a puncture is generated, as the amount of the migrated substance is greater.

Another of the problems caused by the “transuding” or migration of the inner component is encapsulation, in other words, hardness created by the irritation of the scar or wound remaining after the intervention in contact with these transmigrated particles from inside the prosthesis, causing a callosity which hardens the breast.

On the other hand, the weight of the prosthesis significantly increases the natural weight of the breast, therefore with time, the latter tends to sag and require new aesthetic operations, including replacement of the prosthesis, with the aforementioned subsequent drawbacks.

### DESCRIPTION OF THE INVENTION

The breast prosthesis object of the present invention tries to prevent or lessen all these drawbacks, to do so, it is a new implant consisting of a mono-block silicon foam body with or without a covering, which is like a sponge with a gelatin consistency on its surface for providing a soft and natural texture and with a greater density in its nucleus to provide consistency to the compression and recovery of form due to body weight.

Furthermore, this prosthesis provides advantages such as the impossibility of punctures or small ruptures when handled, since, although these do occur, since they lack liquid, the extravasation thereof for any reason is impossible, given that its content is “solid” in a trabecular or micro-cell arrangement, therefore preventing that, with time, this prosthesis loses volume, with its corresponding maintenance, costs, second operations, discomforts, etc.

Since it incorporates no fluid content, all those drawbacks derived from any product more or less irritating for the organism upon bursting disappear, eliminating callosities and hardness in the scar and wound due to irritation with products such as liquid or gelatin silicon, PVP-polyvinylpyrrolidone, saline, hydrogel, triglycerides, etc.

Since it is not a solid implant either and barely contributes to increasing the weight of the woman's breast, sagging of the feminine bust with years is much less than with current implants, having, at least, a much greater weight/volume ratio than the new proposed implant, which have a weight/volume ratio that is equal to or greater than the density of water per unit volume.

This implant is mono-block and mono-substance, in which there is only low density vulcanized silicon, which provides a high radiotransparency, significantly improving the high opacity of the current silicon gel implants; it therefore aids in observing this area of the patient by means of x-rays.

In turn, as vulcanized silicon, which is admitted for medical use by all current international standards (F.D.A., EC, etc.), is the only substance used, implying no risk, it implies approval by health institutions.

### DESCRIPTION OF THE DRAWINGS

To complement the description being made and for the purpose of helping to better understand the features of the invention, a set of drawings accompany the present specification as an integral part thereof which, with an illustrative and non-limiting character, show the following:

FIG. 1. Shows a profile elevational view of the prosthesis object of the present invention.

FIG. 2. Shows a profile cross section view of the prosthesis of the previous figure.

FIG. 3. Shows a profile elevational view of the prosthesis object of the present invention with a filling nucleus.

FIG. 4. Shows a profile elevational view of a conventional prosthesis covered with a layer of the prosthesis object of the present specification.

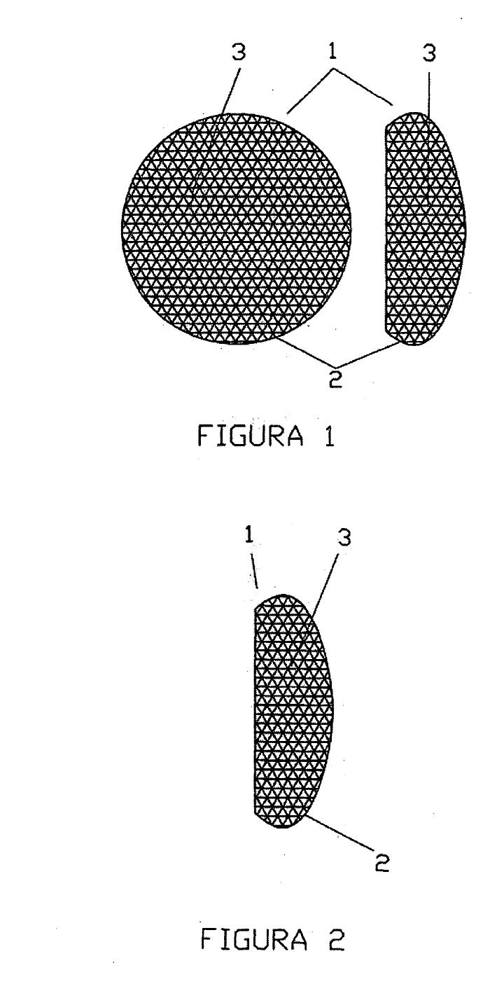

### PREFERRED EMBODIMENT OF THE INVENTION

As can be seen in the attached drawings, first a body is distinguished (1), wrapped or not in a silicon covering (2) which is composed of a trabecular or micro-cell structure, forming a solid and at the same time hollow structure.

FIG. 2 shows that a view of the profile of the prosthesis without a covering coincides with the cross section view of said profile, since the cross section shows the same figure, like a sponge, in other words, an infinity of solidly joined micro-cells (3), covered or not with a silicon covering (2).

Therefore, any sharp object penetrating inside the body of the prosthesis will not burst any membrane containing fluids inside, but rather, at most, it would scratch or perforate some walls of some micro-cells (3), then there is no product that can extravasate from its housing, thus preventing the decrease of volume as well as the encapsulation effect.

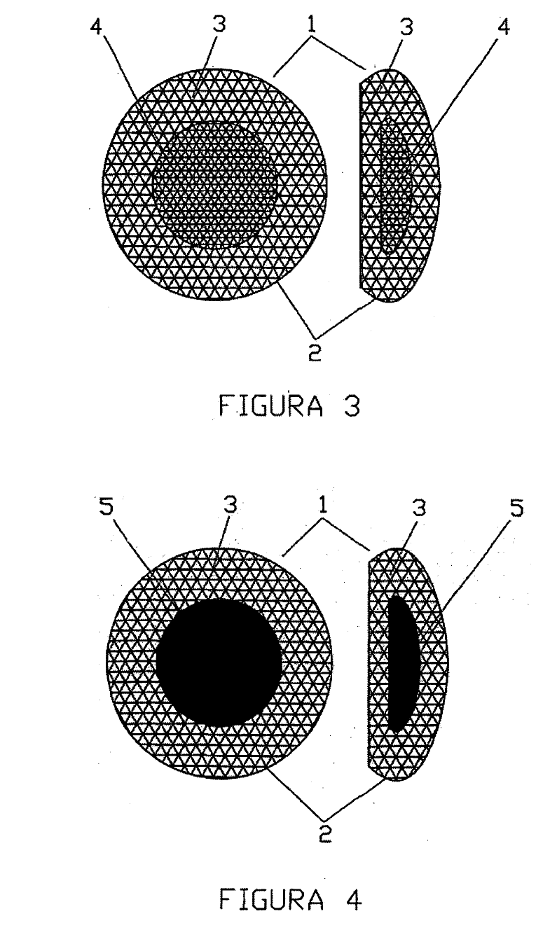

At the same time, to prevent the prosthesis from being crushed by body weight or pressure, this prosthesis is susceptible to the fact that the density of the material used adopts a gradient of lesser to greater density from the outside towards the inside (FIG. 3), such that the outer part has a more or less soft and smooth texture, and the inner part (4) has a higher weight and density, therefore this inner part will be much more difficult to crush, maintaining a volume minimum in the prosthesis regardless of it being crushed.

The object of the invention proposes another solution to crushing, which consists of the inside being a nucleus (5) of gel or saline or any other compound of those currently used, which would imply a covering of a spongy material based on micro-cells (3) over a conventional filled nucleus (5) (with gel, saline, etc.), preventing problems by punctures, by the incision of objects and by extravasation of the inner fluid.

From the aforementioned, a compact but at the same time lighter silicon material breast prosthesis is deduced, whenever the inside of the micro-cells (3) will preferably be hollow, in other words, incorporating air, being susceptible to introducing another compatible gas or substance.

Having sufficiently described the nature of the present invention, as well as an embodiment, all that is left to add is that it is possible to introduce changes of shape, materials and arrangement in its assembly and parts composing it as long as said alterations do not significantly change the features of the invention which are claimed below.

1. A breast prosthesis of those which are introduced in the breast of a woman for aesthetic purposes, characterized in that it consists of a body (1), wrapped or not in a silicon covering (2) which is composed of a trabecular or hollow micro-cells (3) structure, forming a solid and at the same time hollow structure whose material is of silicon, the density of the prosthesis being constant.

2. A breast prosthesis of those which are introduced in the breast of a woman for aesthetic purposes, characterized in that it consists of a body (1), wrapped or not in a silicon covering (2) which is composed of a trabecular or hollow micro-cells (3) structure, forming a solid and at the same time hollow structure whose material is of silicon, varying the density of the prosthesis from lesser to greater from the outside towards the nucleus (4), which also implies a higher trabecular density and weight.

3. A breast prosthesis of those which are introduced in the breast of a woman for aesthetic purposes, characterized in that it consists of a body (1), wrapped or not in a silicon covering (2) which is composed of a trabecular or hollow micro-cells (3) structure, forming a solid and at the same time hollow structure whose material is of silicon, and where the inside is a nucleus (5) of gel or saline or any other compound of those currently used, implying a covering of a spongy material based on micro-cells (3) over a conventional solid nucleus (5).
